(19) United States
(12) Patent Application Publication
(10) Pub. No.: US 2008/0177075 A1
(43) Pub. Date: Jul. 24, 2008

(54) Compositions For Progeny Gender Control

(75) Inventor: Russell Taylor, Berkshire (GB)

Correspondence Address: ROPES & GRAY LLP, PATENT DOCKETING 39/41, ONE INTERNATIONAL PLACE, BOSTON, MA, 02110-2624, US

(73) Assignee: AQ+PLC Stephen Mabbott Associates, Glasgow (GB)

(21) Appl. No.: 11/579,455

(22) PCT Filed: May 3, 2005
(86) PCT No.: PCT/GB2005/001686

§ 371 Date: Nov. 19, 2007

(30) Foreign Application Priority Data
May 4, 2004 (GB) 0409725.9

(51) Int. Cl. C07D 215/26

(52) U.S. Cl. 546/179

(57) ABSTRACT

The present invention relates to the use of a zinc ion chelating agent for the manufacture of a composition for intra-vaginal administration for the control of egg fertilisation by sperm, for progeny gender determination and/or for reducing the incidence of defective progeny.

This invention relates to compositions for use in progeny gender determination, and for modulating intra-vaginal conditions so as to control fertilisation and produce progeny of the desired gender.

For a long time man has longed for the ability to select the sex of offspring so as to maximise or achieve economic and/or other benefits that certain progeny would be able to deliver. In the equine field female horses (fillies) are in much greater demand than male horses (colts) and if it is possible to breed fillies the owners will normally receive a much higher price for them. The same philosophy applies in many cases where the sex of progeny provides certain benefits.

Aristotle suggested that the differences between the sexes were due to the heat of the semen at the time of copulation: hot semen generated males, whereas cold semen generated females. Various experiments and procedures over the years have been tried and the most successful one in place at present is the selection and separation of the X and Y bearing Chromosome sperm.

In all females the eggs available for reproduction normally contains one X chromosome. The sperm from the male contains either an X or Y Chromosome. This means that the sex of the progeny is controlled by the sperm. If an egg is fertilised with an X chromosome sperm then you have a XX embryo and you have a female. If the egg is fertilised by a Y chromosome bearing sperm you then have a XY embryo, which is a male.

There has been work done where the sperm is coloured with a dye and then sorted through a laser sorter that differentiate the sperm based on the difference in colour due to X or Y chromosome. This is due to the fact that the X chromosome sperm contains some 2.8 to 7.5% more DNA than the Y chromosome male producing sperm. This has been successful in many areas but it is time consuming and cumbersome.

Within the X and Y Chromosomes are located two genes. Dax 1 and SRY. (SRY stands for sex-determining region Y gene). SRY is found in the Y chromosome and in the cell it binds to other DNA and in doing so distorts it dramatically out of shape. This alters the properties of the DNA and likely alters the numbers of genes, leading to testis formation. The SRY gene has two zinc fingers as part of its construction and it is thought that these two zinc fingers play an important part in developing the male characteristics.

The SRY gene can also be traced as the potential source of various abnormalities that may arise in the progeny. The DAX 1 gene is associated with the X chromosome and has no zinc fingers and thus has different properties. It is also known that semen is zinc ion rich.

It is an object of the present invention to avoid or minimize one or more of the above problems.

More particularly, it is an object of the present invention to control the level of the zinc ions, the level of other metal ions present and other conditions present with the sperm so as to control or improve the production of the desired progeny.

There has previously been proposed in our earlier Patent publication WO 03/032944, various topical chelating compositions suitable for use in combatting antibiotic-resistant infections and contamination of the skin and open wounds. There has been no previous suggestion, however, of the possible utility of such compositions for progeny gender determination.

We have found that compositions based on the use of chelating compounds which are described hereafter have the properties of chelating zinc ions present and in doing so are able to control the sex of the progeny produced and achieving the desired result.

We have now found that compositions based on the use of zinc ion chelating compounds, including chelating compounds as disclosed in WO 03/032944 (the contents of which are hereby incorporated herein by reference thereto), can influence the fertilisation of eggs by semen. More particularly we have found that the sex of the progeny of mammalian and other animal species, produced through the fertilisation of eggs with sperm, can be controlled. We have also found that fertilisation of eggs by defective sperm, can be inhibited to a greater or lesser degree. We have further found that with a zinc ion chelating agent used with different pH values—from an acidic condition to an alkaline condition, control of the X and Y chromosome sperm is possible and thereby control of the progeny's sex becomes possible.

Thus in a first aspect the present invention provides the use of a zinc ion chelating agent for the manufacture of a composition for the control of egg fertilisation by sperm.

In a second aspect the present invention provides the use of a zinc ion chelating agent for the manufacture of a composition for progeny gender determination through control of egg fertilisation by sperm.

In a third aspect the present invention provides the use of a zinc ion chelating agent for the manufacture of a composition for reducing the incidence of defective progeny through control of egg fertilisation by sperm.

In general it appears that the zinc ion and the zinc ion chelating agent under alkaline conditions, form together a stable complex such that the zinc ion is effectively removed for a sufficient period of time to prejudice the Y chromosome sperm so that it cannot fertilise an egg, with the result that the X chromosome sperm has no competition in fertilising an egg, whereby a female offspring is obtained.

Under acidic conditions, it is not as yet clear, what the actual nature of the interaction with the sperm is, but the effect obtained is a selective inhibition of the X Chromosome sperm (and apparently also defective sperm), so that there is an increase in the that a healthy Y chromosome sperm is able to fertilise the egg, whereby the prospect of obtaining a (sound) male offspring is significantly increased. Without in any way wishing to restrict the scope of the present invention, it is suspected that chelation of ferric ions by a zinc chelating agent which also has a ferric ion chelating capacity—which is generally the case (or indeed by a ferric ion chelating agent which need not necessarily have a zinc ion chelating capacity), may be instrumental in obtaining the effects which we have found.

Another significant benefit of using a chelating compound to achieve this result, is that bacterial infections would be minimised, as infections due to bacteria are metal ion dependent. This is important because successful insemination can be compromised—on the one hand by pre-existing vaginal infections, and on the other hand by infections introduced with the semen. Thus, for example PRRV (Porcine Reproductive and Respiratory Disease) a disease that will decimate any litter within a few weeks of birth is transmitted through Boar's semen. Another example is the Blue Tongue problem with cattle and sheep where a certain amount of the disease is transmitted through semen. Also even if conception is achieved, vaginal infection can result in spontaneous abortion. More particularly we have found that a significant number of spontaneous abortions occurs in pregnant mares with Trichomonas infections, and the incidence of such abortions in infected mares can be substantially reduced when the compositions of the present invention are used. Thus a further benefit of using the present invention is in controlling any infection whether it is from the male's penis upon insertion or from urinal tract infections from other sources. It also ensures that the male's penis is also cleaned of any bacterial infections, after insertion and providing the semen. Thus in a further aspect the present invention also provides the use of a zinc ion chelating agent for the manufacture of a composition for reducing the risk of spontaneous abortion in mares with microbial infections, and especially Trichomonas infections. Preferred chelating agents can chelate various different metal ions in addition to zinc ions, and thereby effectively attack any microbes which may be present in the vagina, thereby reducing the risk of infection of the female during the insemination procedure, and/or reducing any possible prejudice to the fertilisation of the egg and/or initial development of the fertilised egg, as a result of pre-existing vaginal infection of the female. 8-hydroxyquinoline has been found to have a particularly broad spectrum of activity, chelating most metals apart from sodium, potassium and calcium, and thereby be particularly effective against a wide range of microbial infections.

By providing a metal ion chelating agent that removes a variety of metal ions, which may conveniently be referred to as the target metal ions, the effectiveness of the compositions of the invention against microbial infections may be increased. Thus, it is preferable for the metal ion chelating agent to form a chelate with a plurality of metal ions selected from Mg²⁺, Fe²⁺, Cu²⁺, Zn²⁺, Mn²⁺, Ni²⁺, and Se²⁺.

Typically the zinc ion and the zinc ion chelating agent form together a stable complex such that the zinc ion is effectively removed for a sufficient period of time to allow fertilisation to occur, before the zinc ion chelate dissociates. In general the zinc ion and zinc ion chelating agent should form a stable chelate under natural or artificial insemination conditions, and especially intra-vaginal conditions.

Preferably the zinc ion chelating agent is a heteropolar compound comprising at least one unsaturated heterocyclic six-membered ring in which at least one heteroatom moiety acts as a hydrogen acceptor and in which said compound also comprises at least one hydrogen donor moiety, conveniently a hydroxyl group, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound.

In general the preferred zinc ion chelating agent is a hetero aryl compound having at least one nitrogen in the ring structure and at least one hydroxyl substituent disposed on the ring structure so as to provide together, a chelating function. Preferred zinc ion chelating agents are selected from optionally substituted 2,3-dihydroxypyridine; 4,6-dihydroxypryrimidine; 2-pteridinol; 2,4-quinolindiol; 2,3-dihydroxyquinoxalin; 2,4-pteridinediol; 6-purinol; 3-phenanthridinol; 2-phenanthrolinol; 2-phenazinilol, and most preferred is 8-hydroxyquinoline. 8-hydroxyquinoline has the advantage of forming metal ion chelates with a particularly broad range of different metal ions.

In a further aspect the present invention provides an intra-vaginal pharmaceutical composition suitable for use in the intra-vaginal control of fertilisation, said composition comprising a physiologically acceptable zinc ion chelating agent and a pharmaceutically acceptable carrier therefor, in which composition said zinc ion chelating agent has a zinc ion chelating capacity.

The compositions of the present invention for intra-vaginal application in the vagina will generally comprise from 0.0031% to 0.05% w/w, preferably from 0.025 to 0.05% w/w of the chelating agent.

Various forms of composition may be used in accordance with the present invention. Preferably a paste based on an aqueous based liquid composition would be used so as to be retained in the vagina until service has been completed.

In the case of artificial insemination the zinc chelating compound is placed in with the collected sperm and allow to react with the various zinc ions present in the sample before being intra-vaginally introduced in order to fertilise the egg.

It will be appreciated that the choice of components of the composition may be limited by the nature of the zinc ion chelating agent. For example the preferred zinc ion chelating agent 8-hydroxyquinoline is generally insoluble or only poorly soluble in aqueous solution. Suitable aqueous based compositions can be prepared by using an intermediate diluent such as a polyol, including glycols, preferably propylene glycol, glycerine, or sorbitol, and a wetting agent. Those skilled in the art will appreciate that a wide range of wetting agents are available that may be used which would give solubility of the zinc ion chelating agent in glycol, including inter alia Polyoxyethylene Sorbitan Fatty Acid Ester T20, T40, T60 and T80 (Polysorbate), and C9-C11 Alcohol ethoxylate (such as Symperonic 91/8, or, most preferably, Symperonic 91/6).

It will be appreciated that a range of different proportions of the various components of the aqueous based compositions may be used depending on the solubilities of the zinc ion chelating agents used, the final concentration required etc. In general we have found that the amount of wetting agent used is relatively sensitive. In the case of the intermediate diluent (glycol etc), once a required minimum amount sufficient for solubilisation of the zinc ion chelating agent in the water is present, then the amount of this intermediate diluent can be readily increased further, though there is normally no particular advantage in doing so.

In the case of 8-hydroxyquinoline we have found that suitable proportions which may be used in a liquid, aqueous-based, composition of the invention suitable for the control of egg fertilisation by sperm, would in general have the following composition:

| Component | Parts by Weight |
|---|---|
| Zinc ion chelating agent | 1 |
| Wetting agent | 4 +/− 5% |
| Intermediate Diluent | at least 20, preferably 40 |
| De-ionised Water | as required to obtain the |
| | final concentration |
| | required. |

The pH of the composition as described above, is generally in the region of 5.5

In the case where an alkaline pH formulation is required, then the composition would include a suitable additional component, which preferably would be as follows:

| | |
|---|---|
| pH Controller | as required to achieve a |
| | pH of 9.2 to 9.4 |

In the case where a Gel and Paste formulation is required, then the composition would include a suitable additional component as follows:

| | |
|---|---|
| Thickener | as required for the desired |
| | viscosity. |

Various suitable thickeners are known in the art including inter alia hydroxypropylcellulose thickeners such as that commercially available under the Trade Name KLUCEL® H CS from Aqualon of Wilmington, Del., USA. Conveniently there is used a Dehydroxanthan Gum anionic polymer thickener such as that commercially available under the Trade Name AMAZE® XT from National Starch & Chemical Company of Manchester, England. Another alternative suitable for use in the acidic pH compositions, is an acrylic acid polymer thickener such as CARBOPOL® 940 available from Noveon Inc.

For intra-vaginal application the composition is inserted some 20-30 minutes before service occurs by the insertion of 20 mls of the final selected composition by the use of a syringe, to allow sufficient time to ensure that the composition has fully migrated in the vagina and will come in contact with the penis on insertion and also travel with the penis as it is thrust further into the female and comes into contact with the sperm upon ejaculation.

This action of injecting the composition in some time before service will also allow the composition to treat any possible bacterial infections that is present in the vagina and then be ready to treat the sperm when it is ejaculated or otherwise introduced. Treatment of bacteria infections can take from several minutes to approximately one hour with the composition and with it being present in the vagina after service then it will provide the zinc chelating ability to carry out its desired requirement.

In the case of artificial insemination procedures that the collected semen may be treated with a zinc chelating composition and achieve the same end result before the sperm is inserted into the female. This procedure besides chelating the required X or Y chromosome, may also eliminate any bacterially affected sperm before use.

Thus in a further aspect, the present invention provides a method of control of egg fertilisation by sperm in a human or non-human animal comprising the intra-vaginal administration, to an animal, of a composition of the invention.

Further preferred features and advantages of the present invention will appear from the following detailed examples given by way of illustration of some of the preferred embodiments.

### EXAMPLE 1

### Method of Preparation of Concentrate

10 gm of 8-hydroxyquinoline was dissolved at 70 degrees centigrade in 50 grams of a wetting agent selected from Polyoxyethylene Sorbitan Fatty Acid Ester T20, T40, T60 and T80 (polysorbate), and C9-C11 Alcohol ethoxylate (Symperonic 91/6), with 200 grams of water-soluble non-aqueous diluent selected from Propylene Glycol, Glycerine and Sorbitol. Once solution has been achieved, a further quantity of the glycol or glycerine or Sorbitol diluent was added to make up to a solution of 500 grams and the cooled giving 500 grams of 2% w/w 8-hydroxyquinoline concentrate.

### EXAMPLE 2

### Preparation of the X Chromosome Inhibiting Composition

A. Liquid Formulation

Take one part of the 2% w/w 8-hydroxyquinoline concentrate from Example 1 and dilute in 39 parts of de-ionised water. The pH is approximately 5.5 by itself. The strength of this preparation is 500 ppm (0.05%) of 8-hydroxyquinoline and the dosage of 20 mls each time is equivalent to 0.01 ppm (0.0001%) of 8-hydroxyquinoline, which is 100 micrograms per dose. For the use in artificial insemination procedure up to 30 ml of the liquid composition can be placed in the collection tube/bag and after collection ensured that it is well mixed to achieve the desired results.

B. Gel and Paste Formulations

Take one part of the 2% w/w/ 8-hydroxyquinoline concentrate from Example 1 and dilute in 39 parts of de-ionised water. The pH is approximately 5.5 by itself. A dehydroxanthan gum thickener (Amaze XT) is added at the rate of 0.7% w/w for the production the gels and at the rate of 1.5% w/w for the production of the pastes. The strength of this preparation is 500 ppm (0.05%) of 8-hydroxyquinoline and the dosage of 20 mls each time is equivalent to 0.01 ppm (0.0001%) of 8-hydroxyquinoline, which is 100 micrograms per dose.

For the use in artificial insemination procedure up to 30 ml of the liquid composition can be placed in the collection tube/bag and after collection ensured that it is well mixed to achieve the desired results.

### EXAMPLE 3

### Preparation of the Y Chromosome Inhibiting Composition

A. Liquid Formulation

Take one part of the 2% w/w/ 8-hydroxyquinoline concentrate from Example 1 and dilute in 39 parts of de-ionised water, then the pH of this composition is adjusted to 9.2 to 9.7 by the addition of Tetra Sodium Ethylene Diamine Tetra Acetic Acid. The strength of this preparation is 500 ppm (0.05%) of 8-hydroxyquinoline and the dosage of 20 mls each time is equivalent to 0.01 ppm (0.0001%) of 8-hydroxyquinoline, which is 100 micrograms per dose.

B. Gel and Paste Formulations

Take one part of the 2% w/w/ 8-hydroxyquinoline concentrate from Example 1 and dilute in 39 parts of de-ionised water, then the pH of this composition is adjusted to 9.2 to 9.7 by the addition of Tetra Sodium Ethylene Diamine Tetra Acetic Acid. A thickening agent Amaze XT is added at the rate of 0.7% w/w/ for the production of gels and at the rate of 1.5% w/w/ for the production of pastes. The strength of this preparation is 500 ppm (0.05%) of 8-hydroxyquinoline and the dosage of 20 mls each time is equivalent to 0.01 ppm (0.0001%) of 8-hydroxyquinoline, which is 100 micrograms per dose.

1. Use of a zinc ion chelating agent for the manufacture of a composition for the control of egg fertilisation by sperm.

2. Use of a zinc ion chelating agent according to claim 1 for the manufacture of a composition for progeny gender determination through control of egg fertilisation by sperm.

3. Use of a zinc ion chelating agent according to claim 1 for the manufacture of a composition for reducing the incidence of defective progeny through control of egg fertilisation by sperm.

4. Use according to claim 1 wherein said zinc ion chelating agent is a heteropolar compound comprising at least one unsaturated heterocyclic six-membered ring in which at least one heteroatom moiety acts as a hydrogen acceptor and in which said compound also comprises at least one hydrogen donor moiety, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound.

5. Use according to claim 1 wherein said zinc ion chelating agent is a hetero aryl compound having at least one nitrogen in the ring structure and at least one hydroxyl substituent disposed on the ring structure so as to provide together, a chelating function.

6. Use according to claim 5 wherein said zinc ion chelating agent is selected from optionally substituted 2,3-dihydroxypyridine; 4,6-dihydroxypryrimidine; 2-pteridinol; 2,4-quinolindiol; 2,3-dihydroxyquinoxalin; 2,4-pteridinediol; 6-purinol; 3-phenanthridinol; 2-phenanthrolinol; 2-phenazinilol, and 8-hydroxyquinoline.

7. Use according to claim 6 wherein said zinc ion chelating agent is 8-hydroxyquinoline.

8. Use according to claim 1 which includes a wetting agent in said composition.

9. Use according to claim 8 wherein the wetting agent is selected from Polyoxyethylene Sorbitan Fatty Acid Ester T20, T40, T60 and T80 (Polysorbate), and C9-C11 Alcohol ethoxylate (including Symperonic 91/8, and Symperonic 91/6).

10. Use according to claim 1 wherein said composition contains an intermediate solvent in the form of a non-aqueous water soluble solvent, in said composition.

11. Use according to claim 10 wherein said intermediate solvent is a polyol.

12. Use according to claim 11 wherein said intermediate solvent is selected from monoethylene glycol, propylene glycol glycerine, and sorbitol.

13. Use according to claim 1 wherein is included a thickener in said composition.

14. Use according to claim 13 wherein said thickener is a hydroxypropylcellulose thickener.

15. Use according to claim 13 wherein said thickener is a dehydroxanthan gum thickener.

16. Use according to claim 1 comprising 1 part by weight of 8-hydroxyquinoline, 4±5% parts by weight of wetting agent, at least 20 parts by weight of glycol, and water, in said composition.

17. Use according to claim 1 wherein said composition is in the form of a liquid, spray, cream, gel or paste.

18. Use according to claim 1 in which said zinc ion chelating agent is present in the composition at a concentration of from 0.0031% to 0.05% w/w of the chelating agent.

19. Use according to claim 18 in which said zinc ion chelating agent is present in the composition at a concentration of from 0.025 to 0.05% w/w of the chelating agent.

20. Use according to claim 1 wherein said composition includes a pH controller so that said composition has a pH in the range from 7.5 to 10.

21. Use according to claim 20 wherein said composition includes a pH controller so that said composition has a pH in the range from 9.3 to 9.7.

22. Use according to claim 1 wherein said composition is substantially free of any component which would substantially modify the pH of said composition.

23. A method of control of egg fertilisation by sperm in a non-human animal comprising the intra-vaginal administration, to an animal, of a composition according to any one of claim 1.
